(19) United States
(12) Patent Application Publication
(10) Pub. No.: US 2024/0238011 A1
(43) Pub. Date: Jul. 18, 2024

(54) Anesthetic Tunneling Tool

(71) Applicant: EPI-MINDER PTY LTD, East Melbourne (AU)

(72) Inventors: John Michael Heasman, North Turramurra (AU); Rohan J. Hoare, Dallas, TX (US); Tracy Lee Cameron, Toronto (CA)

(21) Appl. No.: 18/290,251

(22) PCT Filed: May 11, 2022
(86) PCT No.: PCT/AU22/50446

§ 371 Date: Nov. 10, 2023

Related U.S. Application Data
(60) Provisional application No. 63/188,558, filed on May 14, 2021.

(51) Int. Cl. A61B 17/34; A61B 17/00; A61N 1/05

(52) U.S. Cl. CPC A61B 17/3468; A61B 17/3417; A61N 1/0504; A61B 2017/00893; A61B 2017/3413; A61B 2017/3454; A61B 2017/3456

(57) ABSTRACT

A surgical tunneling tool and a method of using the surgical tunneling tool are disclosed. In one embodiment, the surgical tunneling tool includes a flexible shaft including a lumen through which an anesthetic may be delivered subcutaneously. In another embodiment, the surgical tunneling tool includes a sheath disposed on a flexible shaft, the sheath including at least one channel through which an anesthetic may be delivered subcutaneously. The subcutaneous distribution of anesthetic may involve stepwise insertion of the surgical tunneling tool. The surgical tunneling tool may be used for insertion of a medical device into a patient after use of the surgical tunneling tool to anesthetize a subcutaneous pathway for the medical device.

### FIELD OF THE DISCLOSURE

The present disclosure relates to surgical tunneling tools, and, more particularly, to anesthetic tunneling tools.

### BACKGROUND

Surgical procedures occasionally require tunneling tools for insertion of subcutaneous devices. These subcutaneous devices include catheters and electrodes. The subcutaneous tunneling includes creating an incision and passing a tunneling tool through the incision. For example, a first and second incision is made and a tunneling tool is passed from one incision to the other. To insert a catheter, for example, while the tunneling tool is disposed subdermally, the catheter is attached to the tunneling tool and the tunneling tool pulled out through the second incision. As the tunneling tool is pulled out, the catheter is pulled between the incisions into a subcutaneous position.

Insertion of subcutaneous devices can involve varying levels of pain and discomfort for the patient. To alleviate the pain or discomfort, a medical professional typically administers anesthetic to the patient. The medical professional may administer a general anesthetic to the patient or they may provide a localized anesthetic in a large bolus delivery to anesthetize the entire region surrounding the subcutaneous path for the subcutaneous device. However, the general anesthetic may unnecessarily lengthen the recovery time and expose the patient to a higher level of risk. Additionally, the bolus delivery of a localized anesthetic may interfere with a medical professional's insertion and placement of a subcutaneous device, because, for example, the bolus delivery of anesthetic may increase the tunnel size or make it difficult to determine the position of the device due to the localized swelling of the tissue due to the introduction of bolus.

In some circumstances, the tunneling tool is a trocar including a handle to push the trocar through the incision. The trocar may include a lumen or other inner cavity to insert medical devices subcutaneously. The insertion of the trocar can be an uncomfortable process for the patient because the tunneling tool is being inserted through an open incision and displaces skin with a sharp, cylindrical tunneling tool.

### SUMMARY

In embodiments, a surgical tunneling tool according to the disclosed embodiments includes a flexible shaft, defining a first lengthwise axis, and including a lumen disposed along a length of the first lengthwise axis of the flexible shaft having a proximal end and a distal end. The tool also includes a handle disposed at the proximal end of the flexible shaft, and a tip disposed at the distal end of the flexible shaft. The tool further includes at least one orifice, disposed proximate the distal end of the flexible shaft, in fluid communication with the lumen.

In embodiments, a surgical tunneling tool according to the described embodiments includes a flexible shaft, defining a first lengthwise axis, and having a proximal end and a distal end. The tool includes a sheath, defining a second lengthwise axis, having a proximal end and a distal end, and disposed on the flexible shaft. The sheath includes at least one channel disposed along a first length of the second lengthwise axis of the sheath. The tool further includes a handle disposed at a proximal end of the flexible shaft, and a tip disposed at the distal end of the flexible shaft.

### BRIEF DESCRIPTION OF THE FIGURES

It is believed that the disclosure will be more fully understood from the following description taken in conjunction with the accompanying drawings. Some of the drawings may have been simplified by the omission of selected elements for the purpose of more clearly showing other elements. Such omissions of elements in some drawings are not necessarily indicative of the presence or absence of particular elements in any of the exemplary embodiments, except as may be explicitly delineated in the corresponding written description. Also, none of the drawings are necessarily to scale. Further, unless otherwise stated, the features of any one embodiment are capable of being combined with features of the other embodiments, and should be considered within the scope of the present disclosure.

FIG. 1 is a perspective view of a surgical tunneling tool in accordance with the present disclosure.

FIG. 2 is a perspective view of an alternative surgical tunneling tool in accordance with the present disclosure.

FIGS. 3A-3V are alternative embodiments of the surgical tunneling tool of FIGS. 1 and 2.

FIG. 4 is a perspective view of an alternative surgical tunneling tool in accordance with the present disclosure.

FIGS. 5A-5D are alternative embodiments of the surgical tunneling tool of FIGS. 1, 2, and 4.

FIGS. 6A-6F are alternative embodiments of the surgical tunneling tool of FIGS. 1 and 2.

Skilled artisans will appreciate that elements in the figures are illustrated for simplicity and clarity and have not necessarily been drawn to scale. For example, the dimensions and/or relative positioning of some of the elements in the figures may be exaggerated relative to other elements to help to improve understanding of various embodiments of the present invention. Also, common but well-understood elements that are useful or necessary in a commercial feasible embodiment are often not depicted in order to facilitate a less obstructed view of these various embodiments. It will further be appreciated that certain actions and/or steps may be described or depicted in a particular order of occurrence while those skilled in the art will understand that such specificity with respect to sequence is not actually required. It will further be appreciated that certain actions and/or steps may be described or depicted in a particular order of occurrence while those skilled in the art will understand that such specificity with respect to sequence is not actually required. It will also be understood that the terms and expressions used herein have the ordinary technical meaning as is accorded to such terms and expressions by persons skilled in the technical field as set forth above except where different specific meanings have otherwise been set forth herein.

### DETAILED DESCRIPTION

A surgical tunneling tool and anesthetic delivery device is disclosed. Specifically, the anesthetic delivery device is included as part of the surgical tunneling tool. A first surgical tunneling tool of the present disclosure includes a trocar having a lumen through which an anesthetic can be delivered subcutaneously. The subcutaneous delivery of anesthesia improves the patient's comfort from the use of a subcutaneous tunneling tool. A second surgical tunneling tool includes a sheath disposed on the surgical tunneling tool. Instead of a lumen, the sheath of the second surgical tunneling tool includes one or more channels that distribute anesthetic subcutaneously. The subcutaneous distribution of anesthetic may involve step-wise insertion of the surgical tunneling tool (e.g., a plurality of further deeper insertions of the surgical tunneling tool). In such embodiments, the surgical tunneling tool is inserted subcutaneously, a medical professional administers a dose of anesthetic via the surgical tunneling tool, the anesthetic is given time to effect the local tissue, and then the medical professional further inserts the surgical tool and another dose of anesthetic is administered. The surgical tunneling tool is inserted in these partial, step-wise insertions until the subcutaneous path is long enough for the subcutaneous device.

After the surgical tunneling tool anesthetizes a subcutaneous pathway, a medical device, such as an electrode, can be comfortably placed. The surgical tunneling tool may pull the electrode through the subcutaneous pathway similar to a catheter. Alternatively, the surgical tunneling tool can be pulled out of the sheath and the subcutaneous electrode passed through the sheath before removing the sheath and securing the electrode subcutaneously.

As used herein, the term “flexible,” as applied to a material or object, means “capable of bending, without breaking, under pressures exerted, by hand, by the average person,” and is inclusive of resilient materials (e.g., elastically deformable materials, such as spring steel) and materials subject to plastic deformation. Furthermore, materials or objects described as “flexible” may include biocompatible materials.

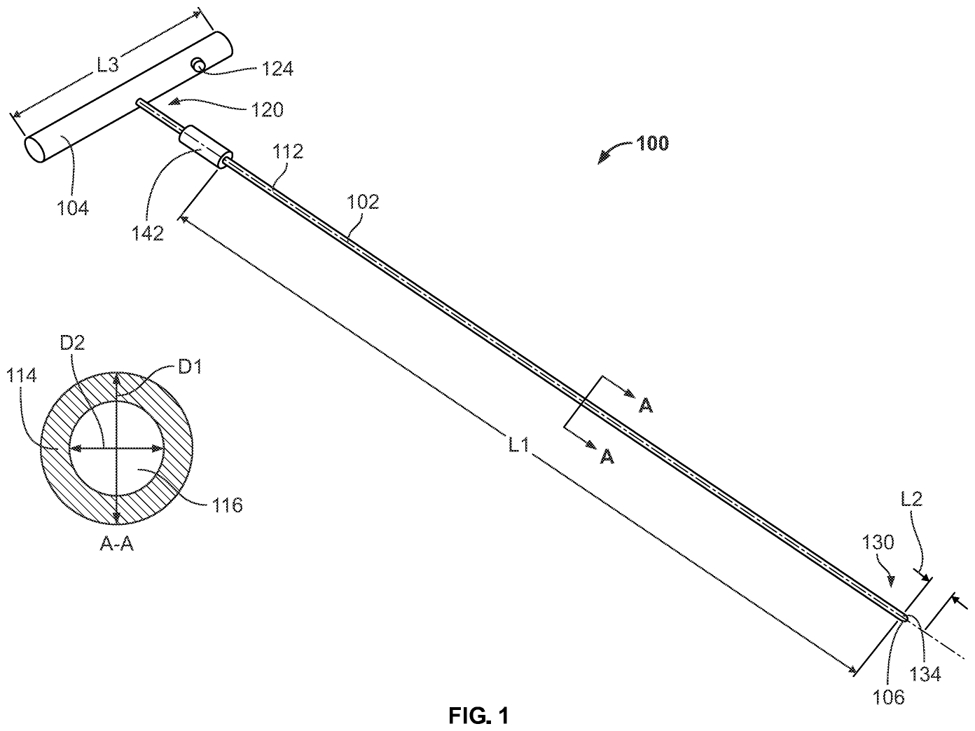

FIG. 1 is a perspective view of a surgical tunneling tool 100 in accordance with the present disclosure. The surgical tunneling tool 100 of FIG. 1 includes a flexible shaft 102, a handle 104, and a tip 106. As shown, the flexible shaft 102, the handle 104, and the tip 106 can be manufactured as a unitary surgical tunneling tool 100 or as separate components. In some embodiments, the flexible shaft 102 and/or the tip 106 may include echogenic particles, enabling a medical professional to track the subdermal position of the flexible shaft 102 and the tip 106 using typical ultrasound apparatus. The echogenic particles may be disposed on the surface of the flexible shaft 102 and the tip 106, disposed throughout the flexible shaft 102 and the tip 106, or disposed in or on only one of the flexible shaft 102 and the tip 106. In some embodiments, the surgical tunneling tool 100 is configured to also include a sheath (not shown in FIG. 1) disposed on a portion of the flexible shaft 102.

The flexible shaft 102, as shown, is generally cylindrical defining a lengthwise axis 112 and a first cross sectional area 114. In other embodiments, the flexible shaft 102 may define a rectangular prism, triangular prism, or a high aspect ratio solid or non-cylindrical shaft. The cross sectional area of the flexible shaft 102 is generally uniform along the lengthwise axis 112. In various embodiments, the cross sectional area 114 of the flexible shaft 102 can vary along the lengthwise axis 112, however the composition and shape of the flexible shaft 102 can vary along any axis or portion of the flexible shaft 102. As shown in FIG. 1, the cross sectional area 114 can include a lumen 116. The lumen 116 is disposed along a length of the lengthwise axis 112 of the flexible shaft 102. As shown, the lumen 116 is centrally disposed in the cross sectional area 114, however the lumen 116 can be disposed off-center of the cross sectional area 114. Additionally or alternatively, the flexible shaft 102 can include more than one lumen 116.

The flexible shaft 102 is configured to bend and to maintain a curve. The flexible shaft 102 may be pre-curved to follow the shape of a bone (e.g., a skull, a rib, etc.). The flexible shaft 102 may, for example, be made of at least one of polyvinyl chloride (PVC), silicone, thermoplastic elastomer (TPE), fluoropolymer, metal (e.g., spring steel, titanium, aluminum, etc.), or short-term biocompatible materials. However, in other embodiments, the flexible shaft 102 is made from metal or other flexible medical-grade materials.

The handle 104 is disposed at a proximal end 120 of the flexible shaft 102. In embodiments such as that depicted in FIG. 1, a lengthwise axis of the handle 104 is perpendicular to the lengthwise axis 112 of the flexible shaft 102. In alternative embodiments, the handle 104 may be disposed at any angle relative the flexible shaft 102, including in line (i.e., sharing a same linear direction) with the flexible shaft 102. Additionally, the handle 104 may include an actuator 124, such as a button as shown in FIG. 1. Alternatively, the actuator 124 can include a lever, a switch, a plunger, a trigger, or any other suitable actuation mechanism. The actuator 124 may be configured to activate or actuate a pump coupled to, or even disposed within, the surgical tunneling tool 100. Such pumps may include one or more of an electric pump, a mechanical pump, a pneumatic pump, a peristaltic pump, or a pump that relies on an electric motor to selectively pressurize fluid disposed in the lumen 116.

The tip 106 is disposed at the distal end 130 of the flexible shaft 102. The tip 106 is in fluid communication with the lumen 116. In some embodiments, the tip 106 includes an orifice 134 in fluid communication with the lumen 116 and an external environment (i.e., environment outside of the flexible shaft 102). However, the orifice 134 may be disposed on the flexible shaft 102, proximate the distal end 130, sufficiently near the tip 106 that an anesthetic injected into the tissue via the orifice 134 may act on the tissue impinged or about to be impinged by the tip 106. In various embodiments, the tip 106 and/or the flexible shaft 102 is in fluid communication with the lumen 116, and is porous. The porosity of the porous material may vary along the length of the lengthwise axis 112 of the flexible shaft 102, to evenly distribute fluid along the length of the flexible shaft. The porous material may be made of any bio-compatible material having a porosity sufficient for the administration of fluid. The tip 106 can be any trocar tip, including a Hasson tip, a blunt tip, an eccentric tip, a pyramidal tip, etc.

Additionally, the flexible shaft 102 includes a fluid connector 142, in fluid communication with the lumen 116. The fluid connector 142 is configured to receive fluid from an external fluid source. As shown in FIG. 1, the fluid connector 142 is disposed proximate the handle 104 on the flexible shaft 102, but the fluid connector 142 can be disposed in the handle 104 or anywhere along the flexible shaft 102. The fluid connector 142 provides a connection between an external fluid source and the lumen 116. Accordingly, the lumen extends along a length of the lengthwise axis 112 of the flexible shaft between the fluid connector 142 and the orifice 134.

The fluid connector 142 may include a luer lock, threaded connection, press-fit connection, or flange connection. As a result, the surgical tunneling tool 100 may be coupled in fluid communication with almost any external fluid source. For example, the surgical tunneling tool may be coupled with a syringe via a standard luer lock fluid connector. In such embodiments, actuation of the plunger in the syringe would fill the lumen 116 with fluid and cause the fluid in the lumen 116 to be expelled through the orifice 134.

In one possible use, the surgical tunneling tool 100 is used to place a sub-scalp electrode on a patient. A medical professional (e.g., doctor, nurse, physician assistant) may anesthetize a local region of a person's head, adjacent the skull, and create an incision in the localized region. After the incision is made, the tip 106 of the surgical tunneling tool 100 may be inserted underneath the skin through the incision in the localized region. When the tip 106 is inserted, the medical professional may actuate the actuator 124 to cause fluid to be selectively pressurized within the lumen (e.g., by activation or actuation of a pump) and released through the orifice 134 disposed on the tip 106. Alternatively, the actuator 124 may be disposed separate from the surgical tunneling tool, such that a patient or a medical professional can selectively pressurize the fluid within the lumen such that it is released through the orifice in response to the patient's or medical professional's actions. The fluid disposed in the surgical tunneling tool 100 is, for example, an anesthetic to anesthetize a second region of tissue outside the original anesthetized local region. After the medical professional administers the anesthetic, and the anesthetic has had sufficient time to anesthetize the second region of tissue, the surgical tunneling tool can be advanced further without discomfort to the patient, and the process repeated as necessary until the tunneling tool 100 has created the desired subcutaneous path. In various embodiments, the surgical tunneling tool 100 forms a subcutaneous path from a first incision to a second incision. The surgical tunneling tool 100 can also be configured to administer an additional fluid such as saline or bio-compatible lubricant (e.g., hyaluronic acid, hydroxypropyl methylcellulose, glycerin, etc.) in addition to or in lieu of the anesthetic. Furthermore, in some embodiments of the surgical tunneling tool 100, a first fluid and a second fluid can both be selectively administered through a single orifice. In such embodiments, the surgical tunneling tool 100 may include additional actuators, reservoirs, fluid sources, etc. to control the selective administration of additional fluids.

Additionally or alternatively, the surgical tunneling tool 100 includes a sheath (not shown in FIG. 1) disposed on, and inserted concurrently with, the flexible shaft 102. The sheath may also have a lengthwise axis, parallel with the lengthwise axis 112 of the flexible shaft 102; a proximal end, adjacent the proximal end 120 of the flexible shaft 102; and a distal end, adjacent the distal end 130 of the flexible shaft 102 and proximate the tip 106. The sheath increases the cross-sectional area of the surgical tunneling tool 100. In such embodiments, the flexible shaft 102 may be removed from the sheath after the subcutaneous path is created, leaving the sheath disposed in the subcutaneous path and holding open the subcutaneous path as the flexible shaft 102 is removed, at which point, a subcutaneous electrode may be inserted into the subcutaneous path via the sheath, and the sheath subsequently removed.

In alternative embodiments, the surgical tunneling tool 100 does not include the actuator 124, but rather, the lumen 116 is in fluid communication with a syringe (e.g., via the fluid connector 142). In various embodiments, actuation of a plunger on the syringe pressurizes the fluid, pushing the fluid into the lumen 116 such that the fluid is dispensed via the orifice 134. However, the lumen 116 of the surgical tunneling tool could be coupled fluidically to any external fluid source and pressurized with any known method of pressurizing a fluid.

The surgical tunneling tool 100 is generally dimensioned for the insertion of a subcutaneous medical device. In various embodiments, the overall length of the surgical tunneling tool 100, from handle 104 to tip 106 can be anywhere from approximately 15 centimeters (cm) to approximately 30 cm (e.g., 15 cm, 17 cm, 20.2 cm, 23.8 cm, 32 cm, etc.). As shown, the flexible shaft 102 may have a length L1 and the tip 106 may have a length L2. In various embodiments, the length L2 of the tip 106 may be between approximately 1 millimeter (mm) and 1 cm and the length L1 of the flexible shaft 102 may be between approximately 12 cm and approximately 30 cm. Accordingly, in various embodiments, the length L1 of the flexible shaft 102 and the length L2 of the tip 106 may constitute seventy-five percent (75%) to one hundred percent (100%) of the total length of the surgical tunneling tool 100. Additionally, the handle 104 can have a length L3 that is up to approximately 2 cm, but is preferably between approximately 8 mm and approximately 10 mm. Further, the flexible shaft 102 may have a diameter D1 that, in various embodiments, is between approximately 0.5 mm and approximately 2 mm. As a result, the lumen 116, disposed within the flexible shaft 102, may have a diameter between 0.25 mm and 1.75 mm. In the various embodiments, the diameter D2 of the lumen 116 is always less than the diameter D1 of the flexible shaft 102.

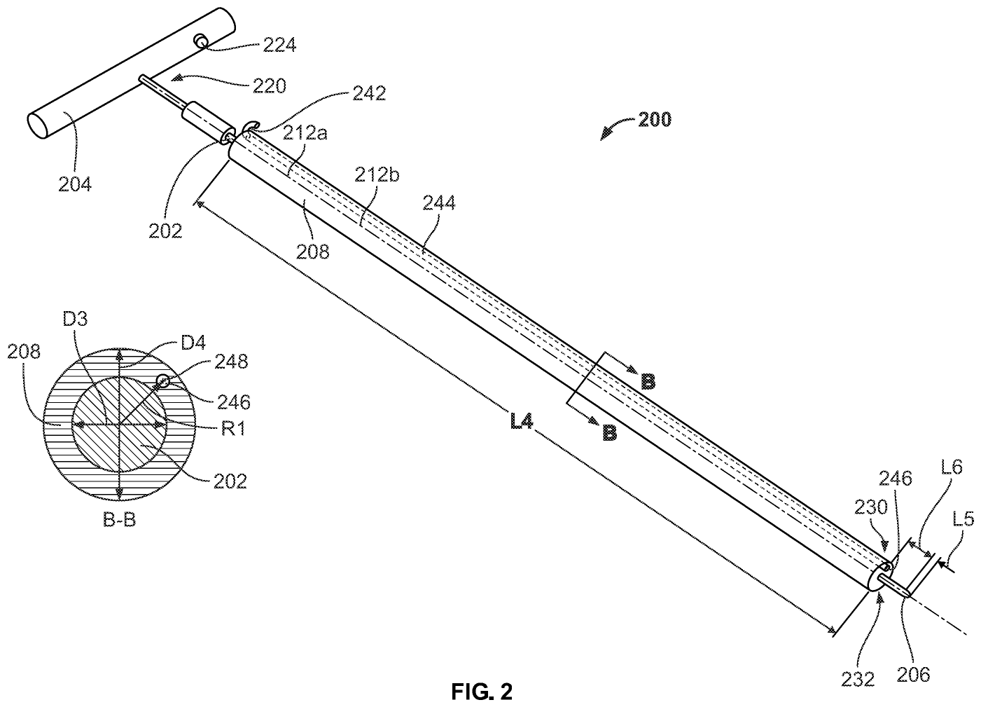

FIG. 2 is a perspective view of a surgical tunneling tool 200 in accordance with an alternative embodiment. As shown, the surgical tunneling tool 200 includes a flexible shaft 202, a handle 204, a tip 206, and a sheath 208. In various embodiments, the flexible shaft 202, the tip 204, and/or the sheath 208 may include echogenic particles, enabling a medical professional to track the subdermal position of the flexible shaft 202 and the tip 206 using typical ultrasound apparatus. The echogenic particles may be disposed on the surface of the flexible shaft 202 and the tip 206, disposed throughout the flexible shaft 202 and the tip 206, or disposed in or on only one of the flexible shaft 202 and the tip 206. Further, the flexible shaft 202, the handle 204, and the tip 206 may be, in various embodiments, identical to the flexible shaft 102, handle 104, and tip 106 of FIG. 1. For example, as with the surgical tunneling tool 100 described above with FIG. 1, the flexible shaft 202, the handle 204, and the tip 206 may be manufactured as a unitary surgical tunneling tool 200 or as separate components.

But, as shown in the embodiment depicted in FIG. 2, the flexible shaft 202 may no longer include a lumen. Instead, the sheath 208 may include at least one channel 210, disposed within the sheath 208 or between the sheath 208 and the flexible shaft 202. The at least one channel 210 disposed within the sheath 208 may be configured for administering a fluid. For example, the fluid may be administered from the distal end 232 of the sheath, proximate the distal end 230 of the flexible shaft 202 and adjacent the tip 206. Similar to the flexible shaft 202, the sheath 208 is also flexible. Additionally, the flexible shaft 202 and the sheath 208 can be configured to bend and, in some embodiments, maintain a curve. In some embodiments, the flexible shaft 202 and/or sheath 208 are bent and maintain a curve of an intended subcutaneous path (e.g., the curve of a skull, rib, etc.). The sheath 208 may be made of at least one of at least one of polyvinyl chloride (PVC), silicone, thermoplastic elastomer (TPE), fluoropolymer, or a short-term biocompatible material. However, in other embodiments, the flexible shaft 102 and/or sheath 208 may be constructed from metal or other flexible medical-grade materials.

The sheath 208 is generally cylindrical and has a lengthwise axis 212a that is parallel with (or, as shown in the embodiment of FIG. 2, coaxial with) the lengthwise axis 212b of the flexible shaft 202, as shown in FIG. 2. The lengthwise axis 212a of the cylindrical sheath 208 additionally defines a cross sectional area that is generally uniform along the lengthwise axis 212a. As shown in FIG. 2, the flexible shaft 202 is disposed within the sheath 208, while in additional embodiments the sheath 208 may be disposed on only a portion of the outer surface area of the flexible shaft 202. In various embodiments, the insertion of the flexible shaft 202 in the sheath 208 may cause the sheath 208 to deform to accommodate the shaft 202. While the flexible shaft 202 has a cross sectional area and the sheath 208 has a cross sectional area, the flexible shaft 202 and the sheath 208 form a combined cross sectional area when the flexible shaft 202 is inserted into the sheath 208. The combined cross sectional area may be larger than sum of the cross sectional area of the flexible shaft 202 and cross sectional area of the sheath 208 if insertion of the flexible shaft 202 into the sheath 208 causes the sheath 208 to deform. As a result, in various embodiments, the combined cross sectional area of the flexible shaft 202 and the sheath 208 may be greater than, less than, or equal to the sum of the cross sectional area of the flexible shaft 202 and the cross sectional area of the sheath 208, depending on how the flexible shaft 202 causes the sheath 208 to deform when the flexible shaft 202 is inserted into the sheath 208.

The handle 204 is disposed at a proximal end 220 of the flexible shaft 202. As shown, the handle 204 is perpendicular to the lengthwise axis 212 of the flexible shaft 202. However, the handle 204 may be disposed at any angle relative the flexible shaft 202. Additionally, the handle 204 may include an actuator 224, such as a button as shown in FIG. 2. Alternatively, the actuator 224 can include a lever, a switch, a plunger, trigger, or other actuation mechanism. The actuator 224 can activate a pump such as an electric pump, a mechanical pump, a pneumatic pump, a peristaltic pump, or a pump that relies on an electric motor to selectively pressurize fluid disposed in sheath 208. In other embodiments, the pump may be couple to or disposed within the surgical tunneling tool 200.

Further, the sheath 208 includes a coupling mechanism 242, in fluid communication with at least one channel 244 disposed in the sheath 208. The coupling mechanism 242 is configured to receive fluid from an external fluid source. The coupling mechanism 242 is disposed on the sheath 208 and proximate the handle 204, but the coupling mechanism 242 can be disposed anywhere along the sheath 208. The coupling mechanism 242 provides a connection between an external fluid source and the at least one channel 244 disposed in the sheath 208. Accordingly, each of the at least one channels 244 extends along a length of the lengthwise axis 212a of the sheath 208 between the coupling mechanism 242 and an orifice 246 disposed on the distal end 232 of the sheath 208.

The fluid connector 242 may include a luer lock, threaded connection, press-fit connection, or flange connection. As a result, the surgical tunneling tool 200 may be coupled in fluid communication with any appropriate external fluid source. Accordingly, the surgical tunneling tool 200 may be fluidically coupled with a syringe via a standard luer lock fluid connector. In such embodiments, actuation of the plunger in the syringe would fill the channel disposed within the sheath 208 with fluid and cause the fluid in the channel to be expelled through the orifice.

The use of the surgical tunneling tool 200 is substantially similar to the use of the surgical tunneling tool 100. A medical professional (e.g., doctor, nurse, physician assistant) will, in accordance with the teachings of the present disclosure, anesthetize a local region of a person's head, adjacent the skull, and create an incision in the localized region. After the incision is made, the distal end 230 and the distal end 232 of the surgical tunneling tool 200 is inserted underneath the skin through the incision in the localized region. When the tip 206 is inserted, the medical professional can actuate the actuator 224. Actuation of the actuator 224 can activate a pump that causes fluid to be pressurized and released through the channel 210 disposed within the sheath 208. In various embodiments, the fluid disposed in the surgical tunneling tool 200 is an anesthetic to anesthetize a deeper portion of tissue than the original anesthetized local region. After the medical professional administers the fluid, the surgical tunneling tool can be further inserted and actuating the actuator 224 is again actuated to administer more fluid. As the surgical tunneling tool 200 is inserted, the surgical tunneling tool 200 creates a subcutaneous path. In some embodiments, the surgical tunneling tool 200 forms a subcutaneous path from a first incision to a second incision.

In one embodiment, after the surgical tunneling tool 200 has fully formed the subcutaneous path, the flexible shaft 202 can be removed from the sheath 208. After the flexible shaft 202 is removed, a subcutaneous electrode can be inserted into the sheath 208. After the subcutaneous electrode is inserted in the sheath 208, the sheath 208 can be removed from the subcutaneous path and the electrode left in the subcutaneous path. Alternatively, the subcutaneous electrode could be disposed on the flexible shaft 202. In such embodiments, the sheath 208 could be removed from the patient, the subcutaneous electrode separated from the flexible shaft 202, and the flexible shaft 202 removed from the patient, leaving the subcutaneous electrode.

The sheath 208 and the flexible shaft 202 are movable relative to each other. As a result, after the surgical tunneling tool 200 is partially or fully inserted, the flexible shaft 202 can be withdrawn while the sheath 208 is kept within a patient. The sheath 208 may retain a cylindrical cross sectional area even after the flexible shaft 202 is removed from the sheath 208 and the sheath 208 is kept within a patient.

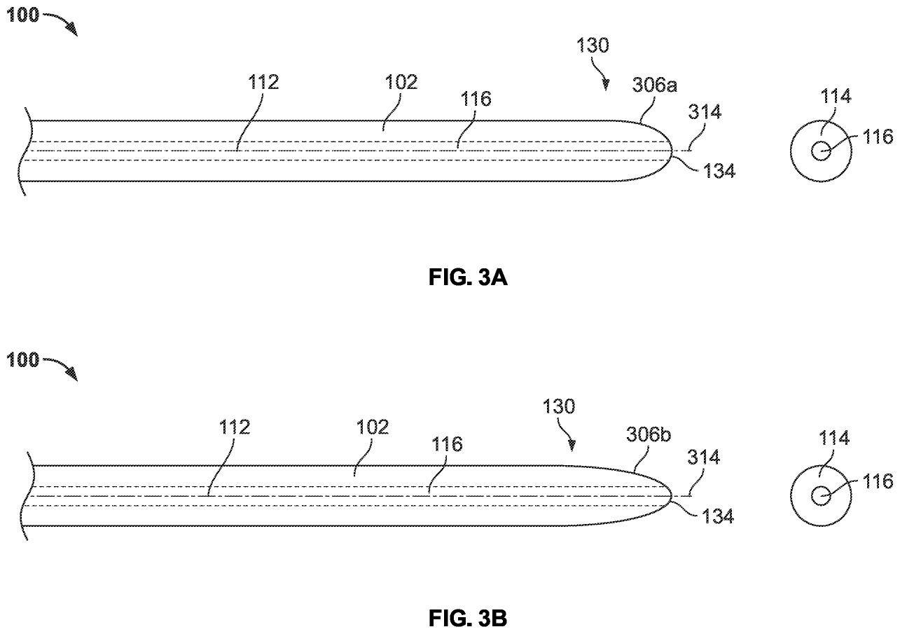

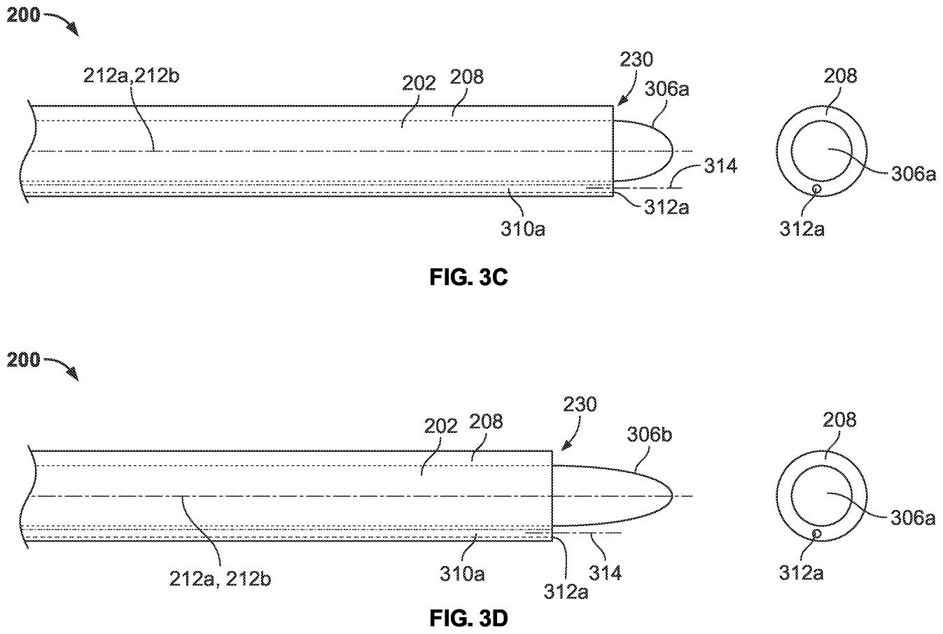

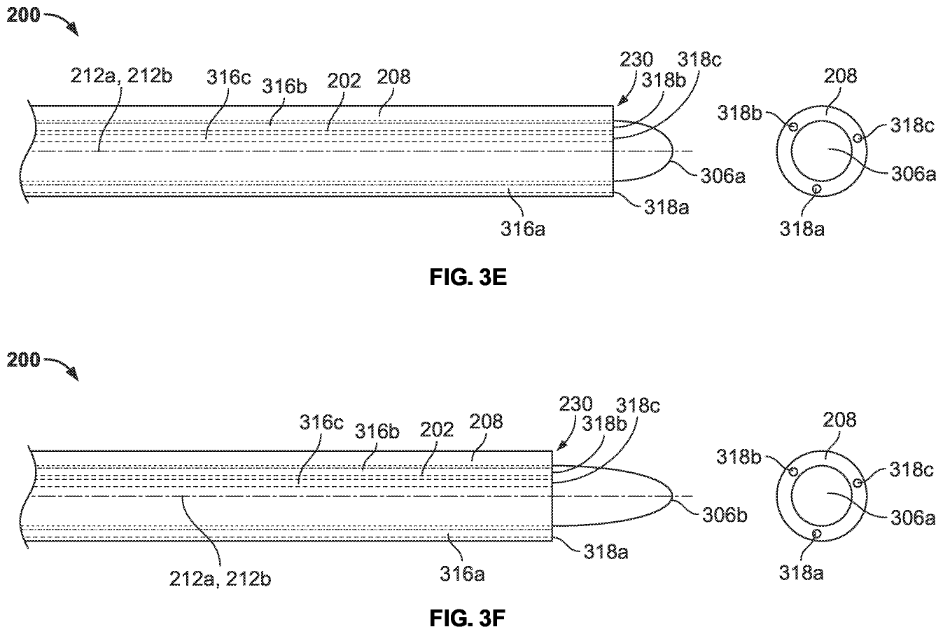

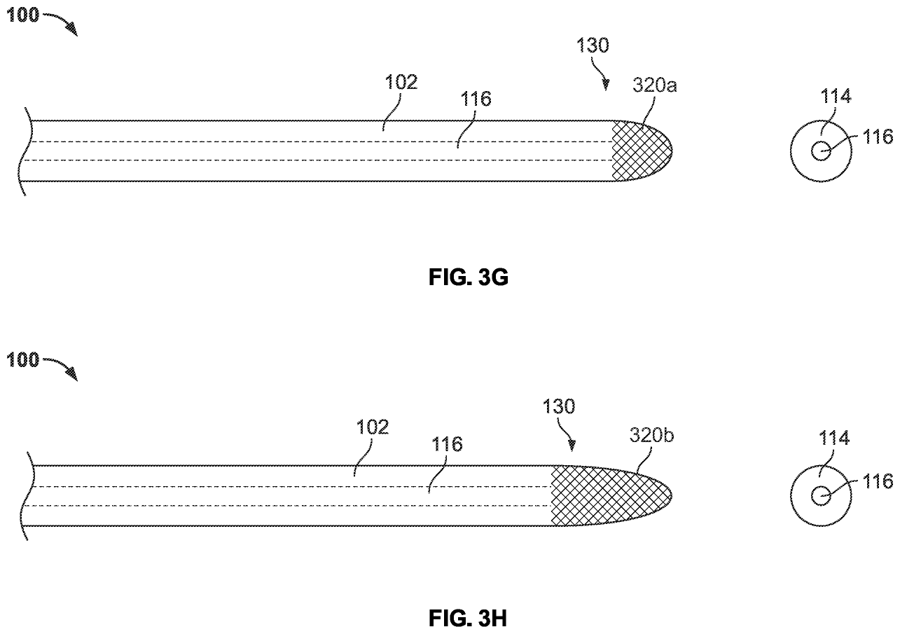

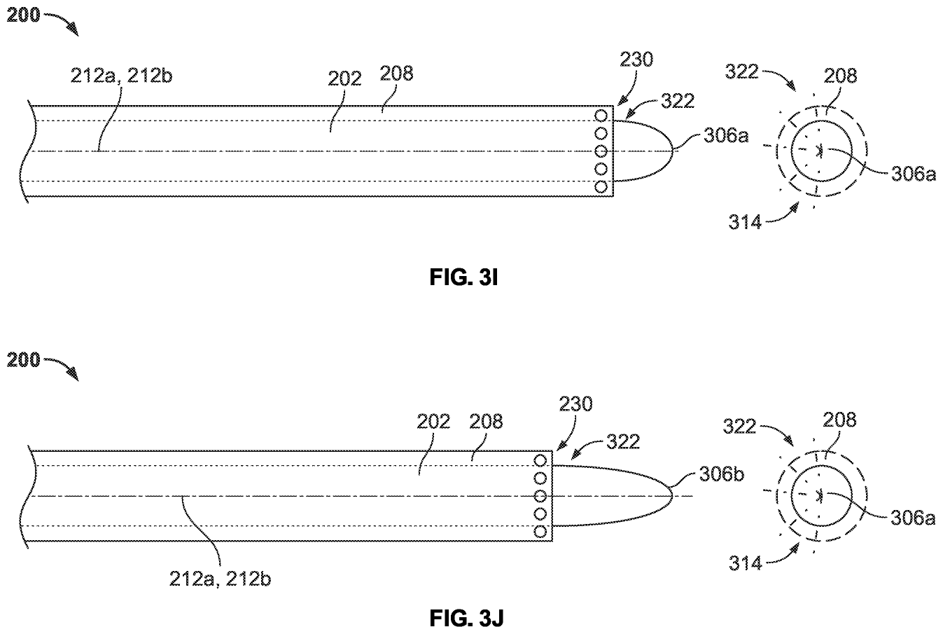

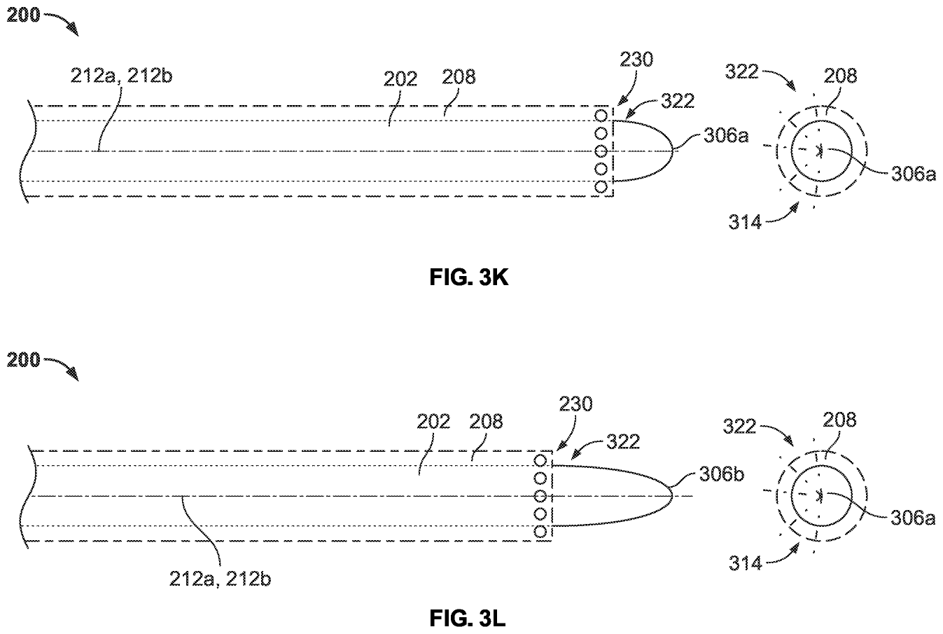

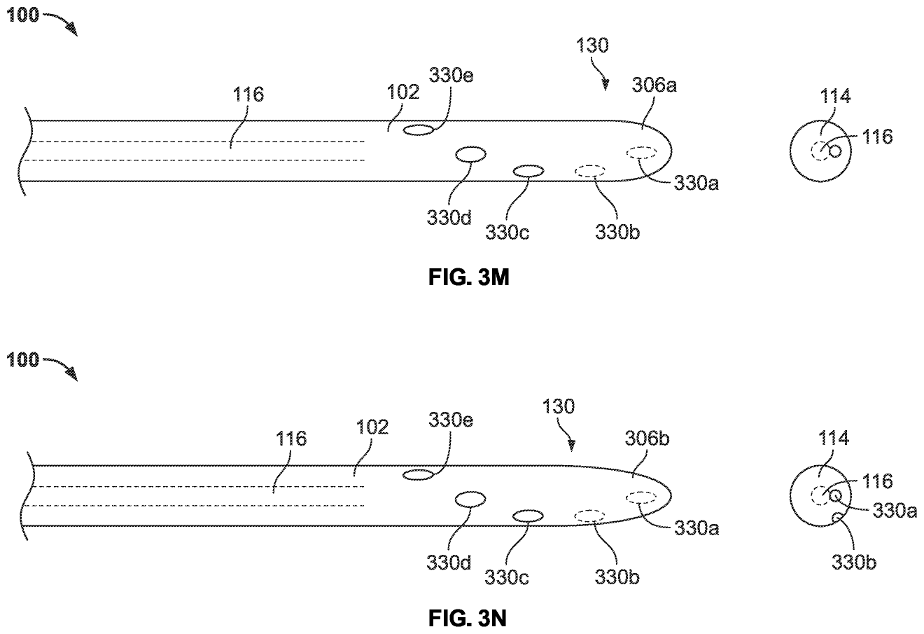

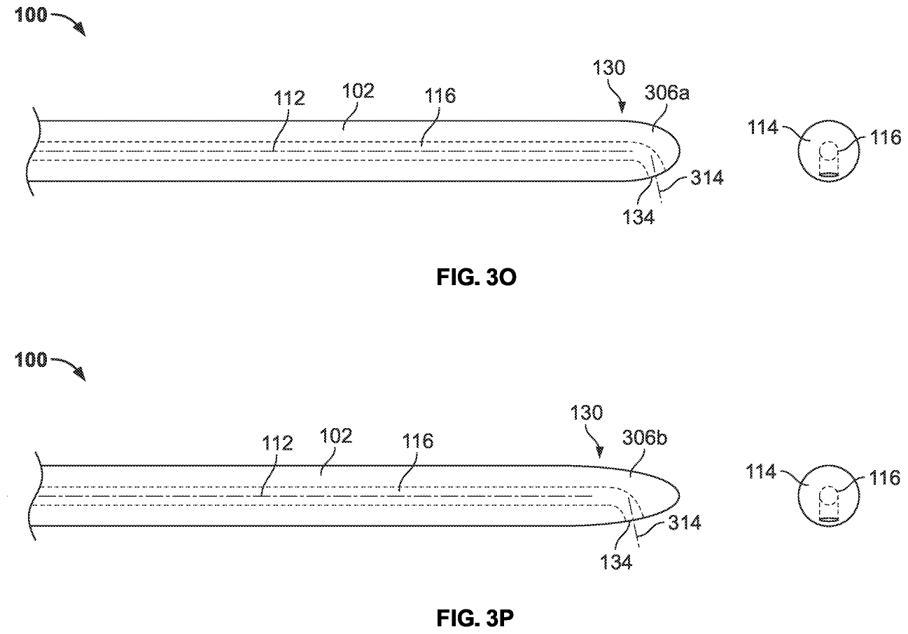

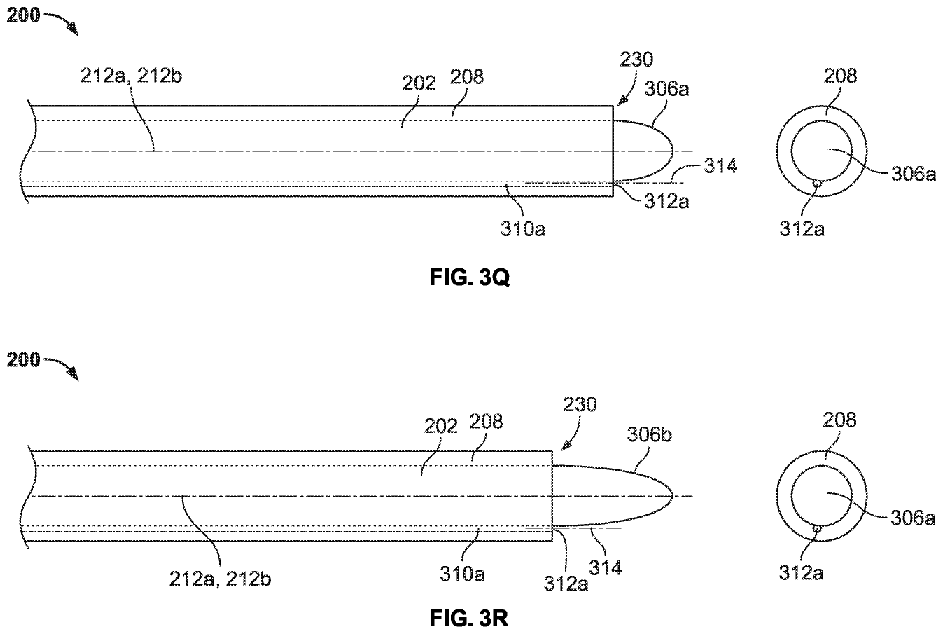

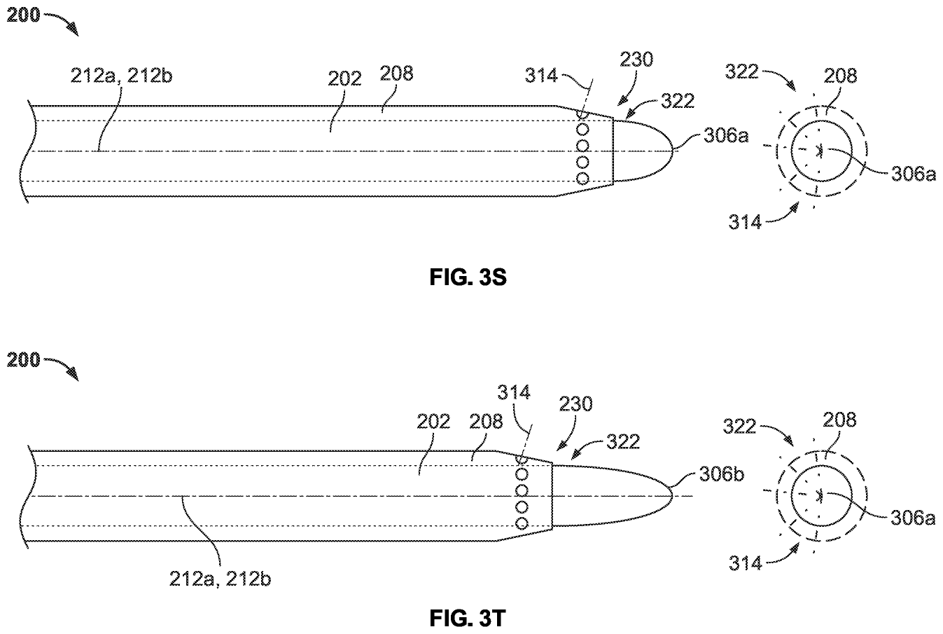

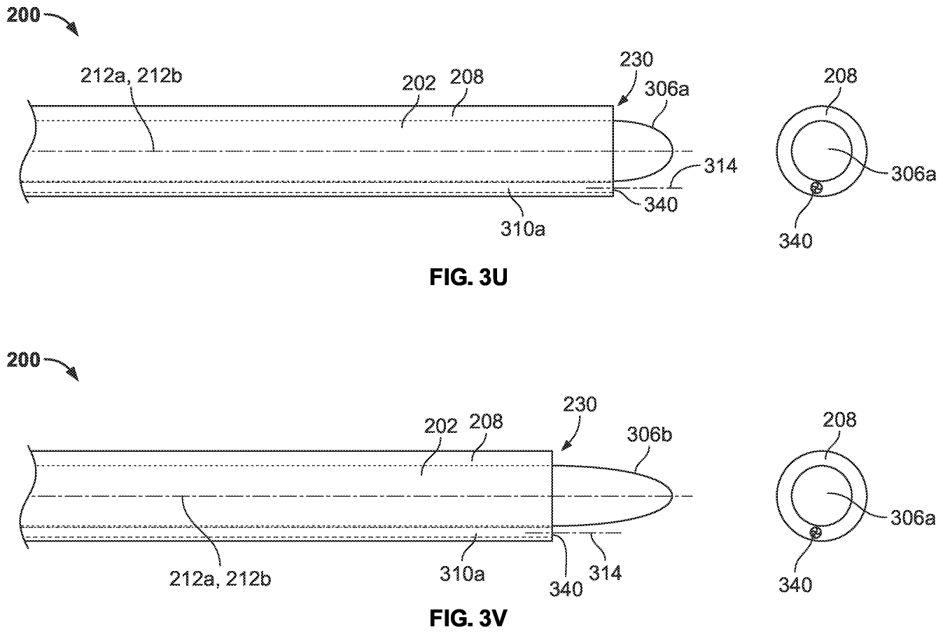

The surgical tunneling tool 200 is generally dimensioned for the insertion of a subcutaneous medical device. In various embodiments, the overall length of the surgical tunneling tool 200, from handle 204 to tip 206 can be anywhere from approximately 15 centimeters (cm) to approximately 30 cm (e.g., 14 cm, 17 cm, 20.2 cm, 23.8 cm, 31.5 cm etc.). As shown, the sheath 208 may have a length L4 and the tip 106 may have a length L5. In various embodiments, the length L5 of the tip 106 may be between approximately 1 millimeter (mm) and 1 cm and the length L4 of the flexible shaft 102 may be between approximately 12 cm and 30 cm. In additional embodiments, the flexible shaft 202 may have a length L6 between the tip 206 and the sheath 208. In such embodiments, the length L6 may be between approximately 0.1 cm and 1 cm. Accordingly, in various embodiments, the length L4 of the sheath 208, the length L5 of the tip 206, and the length L6 of the flexible shaft 202 may constitute seventy-five percent (75%) to one hundred percent (100%) of the total length of the surgical tunneling tool 200. Additionally, the flexible shaft 202 may have a diameter D3 that, in various embodiments, is between approximately 0.5 mm and approximately 2 mm. Accordingly, the sheath 208, disposed about the flexible shaft 202, may have a diameter between 0.75 mm and 3.5 mm. In the various embodiments when the flexible shaft 202 and the sheath 208 are cylindrical, the diameter D3 of the flexible shaft 202 is always less than the diameter D1 of the flexible shaft 102. Furthermore, the channel 244 may include an orifice 246 having a central axis 248 disposed at a radius R1 from the central axis 212a and 212b. In various embodiments, the orifice 246 is disposed at a radius R1 of between approximately 0.4 mm and 1.5 mm. The orifice 246 may also have a diameter between approximately 0.25 mm and 1.75 mm. In various embodiments, the orifice 246 may not be circular and may be disposed between the sheath 208 and the flexible shaft 202 (as shown in FIGS. 3Q and 3R).

FIGS. 3A-3V illustrate alternative aspects of embodiments of the surgical tunneling tools 100 and 200 of FIGS. 1 and 2, respectively. Each of the alternative embodiments provide alternative methods of administering a fluid disposed in the example lumen 116 of FIG. 1 or the example channel 244 disposed in the sheath 208 discussed in connection with FIG. 2.

FIGS. 3A and 3B are alternative embodiments of the surgical tunneling tool 100 in which the lumen 116 is in fluid communication with the orifice 134 disposed on the tip 106. As shown, the orifice 134 is disposed on the furthest end of the surgical tunneling tool 100 and the tip 106, however, the orifice 134 may be disposed anywhere on the tip 106. FIG. 3A is an example surgical tunneling tool 100 having a Hasson tip 306a while FIG. 3B is an example surgical tunneling tool 100 having a blunt tip 306b. In both FIGS. 3A and 3B, the lumen 116 is in fluid communication with the orifice 134 and fluid pressurized in the lumen 116 can pass through the lumen 116 and out the orifice 134. As shown in FIGS. 3A and 3B, the lumen 116 is centrally disposed on the flexible shaft 102 and the orifice 134 is centrally disposed on the tip 106. Additionally, the orifice 134 includes an axis 314, disposed centrally within the orifice 134. As shown in FIGS. 3A and 3B, the axis 314 of the orifice 134 is parallel with the lengthwise axis 112 of the flexible shaft 102. However, the orifice 134 could be disposed on the tip 106 and the axis 314 could be oblique or perpendicular to the lengthwise axis 112 (as shown in FIGS. 3O and 3P).

FIGS. 3C and 3D are alternative embodiments of the surgical tunneling tool 200 in which the sheath 208 includes a channel 310 having an orifice 312 disposed proximate the distal end 230 of the flexible shaft 202. The orifice 312 includes an axis 314, disposed centrally within the orifice 312, parallel with the lengthwise axis 212 of the flexible shaft 202. In the example of FIG. 3C the surgical tunneling tool 200 includes a Hasson tip 306a while FIG. 3D is an example surgical tunneling tool 200 having a blunt tip 306b. As shown in FIGS. 3C and 3D, the flexible shaft 202 does not include a lumen, instead the sheath 208 includes the channel 310. In such embodiments, fluid can be administered from the channel 310 out of the orifice 312.

FIGS. 3E and 3F are also alternative embodiments of the surgical tunneling tool 200. In contrast to the surgical tunneling tool 200 of FIGS. 3C and 3D, the surgical tunneling tool of FIGS. 3E and 3F include a first channel 316a, a second channel 316b, and a third channel 316c. Each of the channels 316a, 316b, 316c have a corresponding first orifice 318a, second orifice 318b, and a third orifice 318c. As shown in FIGS. 3E and 3F, the channels 316a, 316b, and 316c and the orifices 318a, 318b, and 318c are disposed equidistantly about the periphery of the sheath 208 (e.g., spaced approximately 120 degrees apart). Additionally, each of the orifices 318a, 318b, and 318c includes a central axis (not shown) parallel with the lengthwise axis 212b of the flexible shaft 202. Alternatively, the channels 316a, 316b, and 316c and the orifices 318a, 318b, and 318c may be unevenly distributed about the periphery of the sheath 208 (e.g., orifices 318a and 318b are approximately sixty degrees (60°) apart from each other and orifice 318c is approximately one-hundred fifty degrees (150°) apart from both orifices 318a and 318b). In the examples of FIGS. 3E and 3F, fluid disposed in the channels 316a, 316b, and 316c can be dispensed through the orifices 318a, 318b, and 318c, respectively, evenly about the periphery of the sheath. Accordingly, in various embodiments, the fluid can be dispensed through a subset of the channels 316a, 316b, and 316c and the orifices 318a, 318b, and 318c. Additionally or alternatively, the surgical tunneling tool 100 may administer fluid through one of the orifices 318a, 318b, or 318c, administer a second fluid through a second orifice of 318a, 318b, and 318c, and/or suction fluid through a third orifice of 318a, 318b, and 318c. The surgical tunneling tool 100 including an orifice that suctions fluid prevents a subdermal pathway from becoming excessively swollen or flooded.

FIGS. 3G and 3H are alternative embodiments of the surgical tunneling tool 100 of FIG. 1. As shown in FIGS. 3G and 3H, the surgical tunneling tool 100 includes the lumen 116 in fluid communication with a porous tip 320a and 320b, respectively. Alternatively, some or all of the flexible shaft 102 is also made with a porous material. As a result, when fluid disposed in the lumen 116 is pressurized, the fluid passes through the tip 320a and 320b and out of the surface of the tip 320a and 320b. As shown in FIGS. 3G and 3H, the tip 320a and the tip 320b are entirely made of a porous material, however, in some embodiments, the tip 320a and the tip 320b are only partially made of a porous material. In such embodiments, the porous material may be selected and/or configured to optimize distribution and/or flow rate of the fluid to an external environment.

FIGS. 3I, 3J, 3K, and 3L are alternative embodiments of the surgical tunneling tool 200. As shown, the sheath 208 includes a plurality of orifices 322 disposed at a distal end 232 of the sheath 208 proximate the tip 306a or the tip 306b. Each orifice of the plurality of orifices 322 includes an axis 314, disposed centrally within the each orifice of the plurality of orifices 322, disposed perpendicular to the lengthwise axis 212b of the flexible shaft 202. In other embodiments, as shown in FIGS. 3S and 3T, each axis 314 may be disposed obliquely to the lengthwise axis 212b. Returning to the illustrated examples of FIGS. 3I, 3J, 3K, and 3L, the plurality of orifices 322 are evenly distributed about the periphery of the sheath 208, however, the plurality of orifices 322 may be unevenly distributed on the sheath 208. Additionally, the sheath 208 may include more or fewer orifices 322 than shown in FIGS. 3I, 3J, 3K, and 3L. Furthermore, in the illustrated examples, the sheath 208 does not include a channel, however in other embodiments each orifice of the plurality of orifices 322 includes a channel. In such embodiments, a subset of the plurality of orifices 322 may distribute a different fluid and/or may be configured to suction fluid. In yet other embodiments, the sheath 208 may include an open space along a length of the sheath 208 in which fluid is disposed.

FIGS. 3K and 3L are alternative embodiments of the surgical tunneling tool 200 shown in FIGS. 3I and 3J. In the examples of FIGS. 3K and 3L, the sheath 208 is compressible such that pressure exerted on the sheath 208 causes fluid to be released through the orifices 322. In one embodiment, the pressure on the sheath 208 as the surgical tunneling tool 200 is inserted subcutaneously (i.e., the pressure created by the tissue through which the tool 200 is tunneling compressing the sheath 208) is sufficient to release fluid through the orifices 322. As a result, the fluid is released proportional to the insertion of the surgical tunneling tool 208.

FIGS. 3M and 3N are alternative embodiments of the surgical tunneling tool 100. In the example surgical tunneling tool 100, the flexible shaft 102 includes a plurality of orifices 330a, 330b, 330c, 330d, and 330e. In various embodiments, the surgical tunneling tool 100 may include more or fewer orifices. In some such embodiments, the orifices may extend along some, most, or all of the length of the flexible shaft 102. As shown, the orifices 330a, 330b, 330c, 330d, and 330e are disposed in a helical pattern along an outer surface of the flexible shaft 102 and proximate the distal end 130. Additionally or alternatively, the orifices 330a, 330b, 330c, 330d, and 330e may be disposed linearly along a length of the flexible shaft proximate the tip. Each of the orifices 330a, 330b, 330c, 330d, and 330e is in fluid communication with the lumen 116, and the tip 306a, 306b may be hollow to evenly disperse fluid from the lumen through the orifices 330a, 330b, 330c, 330d, and 330e. Additionally or alternatively, the orifices 330a, 330b, 330c, 330d, and 330e are differently sized to control the dispersal of fluid from the lumen. For example, the orifice 330a, nearest the tip 306a or 306b, may be the largest orifice, and orifices successively further from the tip 306a or 306b may be progressively smaller. In such embodiments, the orifices may be sized such that each orifice administers an equal (or, in any event, desired relative) amount of fluid for a given fluid pressure, ensuring, for example, that fluid is dispensed at the tip, despite there being orifices closer to the reservoir. In yet further embodiments, a subset of the orifices may be connected to a lumen different from the other orifices, such that a second fluid may be administered or for fluid to be selectively suctioned away within the subdermal pathway.

FIGS. 3U and 3V depict additional embodiments of the surgical tunneling tool 200. As shown, the surgical tunneling tool 200 is substantially similar to the surgical tunneling tool 200 shown in FIGS. 3C and 3D. The surgical tunneling tools 200 may include a sheath 208 and a channel 310a. However, the surgical tunneling tool 200 includes an orifice having an elastomeric cover 340 (e.g., a polymer membrane). The cover 340 may be a pressure-sensitive, one-way valve. In such embodiments, the cover 340 may only open when pressure in the channel 310a is sufficiently great to open the cover 340 (e.g., pressure differential of 1 pound per square inch (psi), 0.25 psi, 2 psi, 5 psi, 10 psi, etc.). Alternatively, in other embodiments, the cover 340 may be actuated by an opening mechanism disposed on the surgical tunneling tool (e.g., the actuator 224). When the cover 340 is closed, the cover 240 does not leak any fluid. But, when the cover 340 has been opened, fluid disposed in the channel 310a is expelled from the channel 310a.

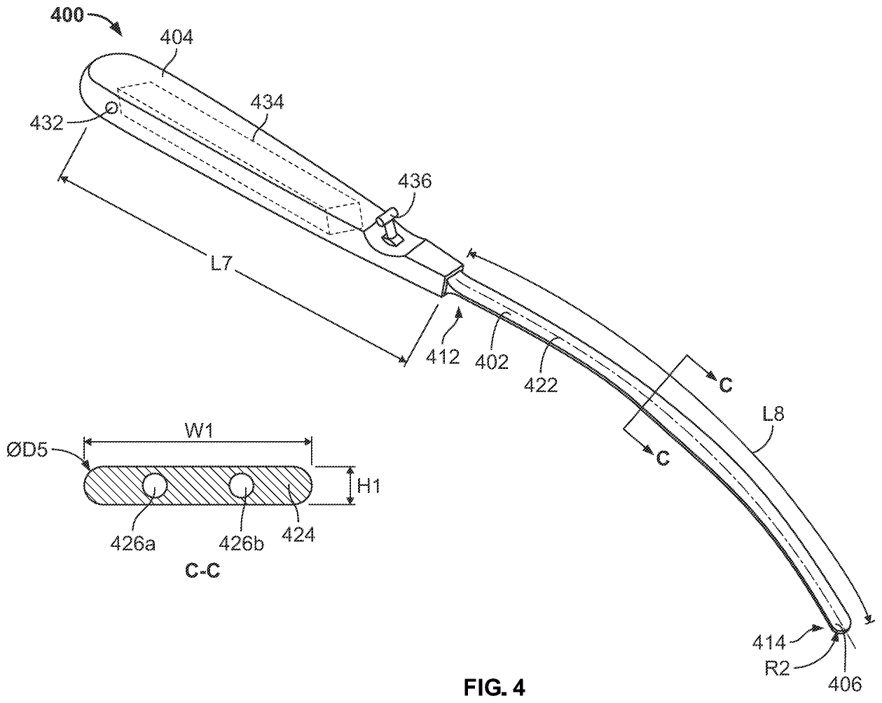

FIG. 4 is a perspective view of a surgical tunneling tool 400 in accordance with the present disclosure. The surgical tunneling tool 400 of FIG. 4 includes a flexible shaft 402, a handle 404, and a tip 406. As shown, the flexible shaft 402, the handle 404, and the tip 406 can be manufactured as a unitary surgical tunneling tool 400 or as separate components. In contrast to the surgical tunneling tools 100 and 200, the surgical tunneling tool 400 has a handle in line with the flexible shaft 402, rather than transverse to the flexible shaft 402. In some embodiments, the flexible shaft 402 and/or the tip 406 may include echogenic particles, enabling a medical professional to track the subdermal position of the flexible shaft 402 and/or the tip 406 using typical ultrasound apparatus. The echogenic particles may be disposed on the surface of the flexible shaft 402 and the tip 406, disposed throughout the flexible shaft 402 and the tip 406, or disposed in or on only one of the flexible shaft 402 and the tip 406. In some embodiments, the surgical tunneling tool 400 is configured to also include a sheath (not shown in FIG. 4) disposed on a portion of the flexible shaft 402.

As shown, the flexible shaft 402 is generally flat with an obround (e.g., parallel sidewalls with a hemispherical end) tip 406. Alternatively, the flexible shaft 402 may have a tip 406 having a different shape, for example, triangular, flat, elliptical, etc. The flexible shaft includes a proximal end 412 and a distal end 414 and having a lengthwise axis 422. Along the lengthwise axis 422, the flexible shaft 402 includes a cross sectional area 424, and as shown, the cross sectional area 424 is obround. In other embodiments, the cross sectional area 424 is rectangular, oblong, elliptical or other similar shape. The cross sectional area 424 can include lumens 426a and 426b. Additionally or alternatively, the flexible shaft 402 may include more or fewer lumens.

The flexible shaft 402 may be configured to bend and, in embodiments, to maintain a curve. The flexible shaft 402 may be pre-curved to follow the shape of a bone (e.g., a skull, a rib, etc.). The flexible shaft 402 may, for example, be made of at least one of polyvinyl chloride (PVC), silicone, thermoplastic elastomer (TPE), fluoropolymer, or metal (e.g., spring steel, titanium, aluminum, etc.). However, in other embodiments, the flexible shaft 402 is made from metal or other flexible medical-grade materials.

The tip 406 is disposed at the distal end 414 of the flexible shaft 402. The tip 406 is in fluid communication with the lumen 416. In some embodiments, the tip 406 includes at least one orifice (not shown in FIG. 4) in fluid communication with either lumen 426a or 426b and an external environment (i.e., environment outside of the flexible shaft 402). However, the orifice may be disposed on the flexible shaft 402, proximate the distal end 414, sufficiently near the tip 406 that an anesthetic injected into the tissue via the orifice may act on the tissue impinged or about to be impinged by the tip 406. In various embodiments, the tip 406 and/or the flexible shaft 402 is in fluid communication with at least one of lumen 426a and/or lumen 426b, and is porous. The porous material may be made of any bio-compatible material having a porosity sufficient for the administration of fluid.

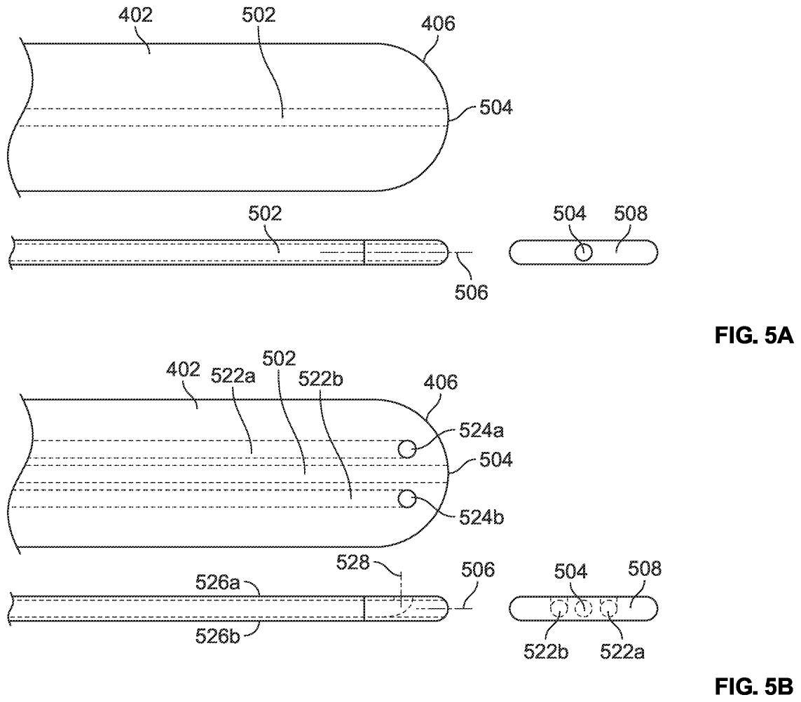

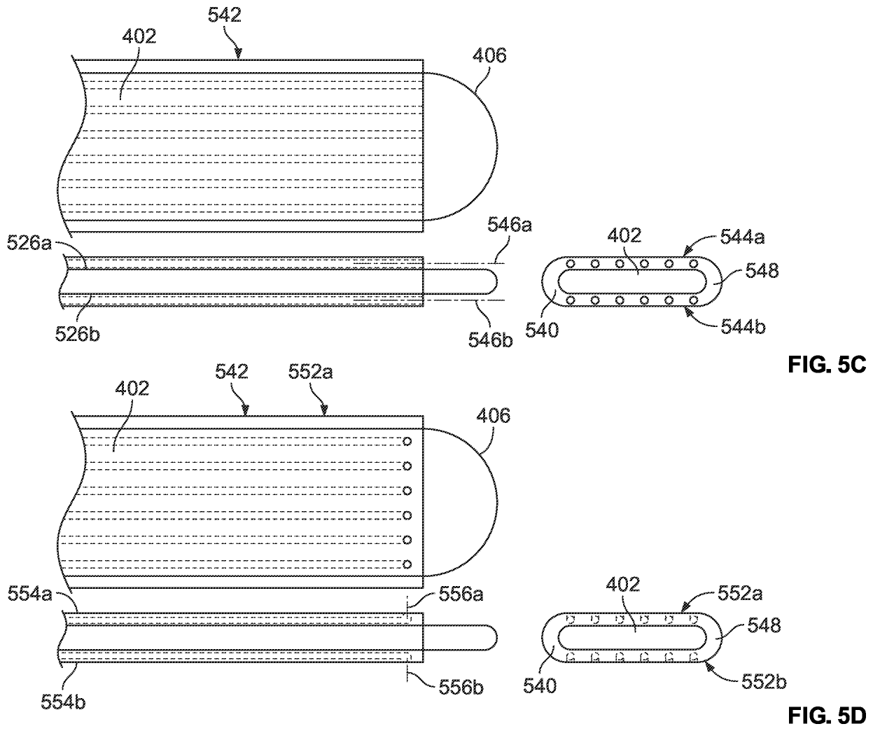

The handle 404, shown in FIG. 4, includes a fluid connector 432, an internal container 434, and a lever actuator 436. In accordance with the present disclosure, the fluid connector 432 is in fluid communication with the internal container 434 and the internal container 434 is in fluid communication with a lumen, such as either one of 426a and 426b, disposed in the flexible shaft 402. Additionally or alternatively, the internal container 434 may be in fluid communication with a channel disposed in a sheath (shown in FIGS. 5C and 5D). In such an embodiment, the handle may include a second fluid connection (not shown) to establish a fluid connection between the internal container 434 and the channel in the sheath. The internal container 434 can operate as an internal fluid source for the surgical tunneling tool 400. Additionally or alternatively, the lever actuator 436 can be operated by a user of the surgical tunneling tool 400 to control dispensing fluid from the internal container 434 through the flexible shaft 402 and to the external environment through an opening disposed the flexible shaft 402 or the tip 406. In yet further embodiments, the handle 404 may include more than one internal container. Accordingly, the containers may include different concentrations of anesthetic or different fluids (e.g., saline, bio-compatible lubricant, etc.). In some embodiments, a higher concentration anesthetic is administered at a tip of the surgical tunneling tool 400, while lower concentration anesthetic is administered along the flexible shaft 402. In yet further embodiments, a first fluid is administered from a first internal container and out a tip of the surgical tunneling tool 400 while a second fluid is administered from a second internal container and out a sheath of the surgical tunneling tool 400.

The surgical tunneling tool 400 is generally dimensioned to facilitate the insertion of a corresponding subcutaneous medical device. In various embodiments, the overall length of the surgical tunneling tool 400, from handle 404 to tip 406 can be anywhere from approximately 20 centimeters (cm) to approximately 40 cm (e.g., 20 cm, 24 cm, 27.2 cm, 35 cm, 42 cm, etc.), in embodiments. As shown, the handle 404 may have a length L7, the flexible shaft 402 may have a length L8, and the tip 406 may have a radius R2. In various embodiments, the length L7 of the handle can be anywhere from approximately 7.5 cm to approximately 15 cm, in embodiments. Additionally, the length L8 of the flexible shaft can be anywhere from approximately 8 cm to 20 cm, in embodiments. Additionally, the tip 406 can be between approximately 0.25 cm to 2 cm in embodiments. Further, the flexible shaft 402 may have a height H1 and a width W1 that. In in various embodiments, the width W1 is between approximately 0.1 cm and 1 cm, and the height H1 is between approximately 0.5 mm and approximately 0.5 cm in embodiments. Further, the lumens 426a and 426b, disposed within the flexible shaft 402, may have a diameter between 1 mm and 3 mm in embodiments. In the various embodiments, the diameter of the lumens 426a and 426b are less than the height of the flexible shaft 402.

FIGS. 5A and 5B are alternative embodiments of the surgical tunneling tool 400, including at least one lumen 502 disposed in the flexible shaft 402. The flexible shaft 402 may include one lumen 502 as shown in FIG. 5A, including an axis 506 perpendicular to the front surface 508 or may include multiple lumens 502, 522a, and 522b as shown in FIG. 5B. Each lumen 502, 522a, and 522b is associated with an orifice 504, 524a, and 524b. As shown in FIG. 5B, one or more orifices 524a and 524b can be disposed on a top surface 526a or a bottom surface 526b of the flexible shaft 402. Additionally or alternatively, the orifices 502, 524a, and 524b may be disposed on the tip, the top surface 526a, and the bottom surface 526b. Further, the orifices 524a and 524b include an axis 528 disposed perpendicular to the top surface 526a. In other examples, any of the axis 506 or 528 may be disposed oblique to the front surface 508, the top surface 526a, and/or the bottom surface 526b.

FIGS. 5C and 5D are alternative embodiments of the surgical tunneling tool 402. As shown, the flexible shaft 402 is disposed within a sheath 540 including a plurality of channels 542. Additionally or alternatively, the sheath 540 may include more or fewer channels 542 than shown in FIGS. 5C and 5D. Furthermore, each of the channels can be in fluid communication with an exterior environment via openings 544a and 544b. As shown, the openings 544a are disposed adjacent the top surface 526a and the openings 544b are disposed adjacent the bottom surface 526b. Additionally, each of the openings 544a can have an axis 546a perpendicular to the sheath 540 front surface 548. Further, the openings 544b can have an axis 546b perpendicular to the sheath 540 front surface 548. Alternatively, as shown in FIG. 5D, the sheath 540 can include a plurality of channels 542, but the openings 552a can be disposed in a top surface 556a of the sheath 540 and openings 552b can be disposed in a bottom surface 556b. As shown in FIG. 5D, the openings 552a can include an axis 556a disposed perpendicular to the top surface 554a and the openings 552b can include an axis 556b disposed perpendicular to the bottom surface 554b. Alternatively, the openings 544a, 544b, 552a, and 552b can be disposed on the sheath 540 such that the axis 546a, 546b, 556a, and 556b are disposed obliquely to the sheath 540.

Furthermore, while the embodiments in FIGS. 3A-3V are shown in connection with a cylindrical flexible shaft 102, 202 and the embodiments 5A-5D are shown with a generally flat flexible shaft 402, the surgical tunneling tools 102, 202, and 402 can be constructed and/or modified in accordance with any of the shown embodiments in FIGS. 3A-3V and 5A-5D.

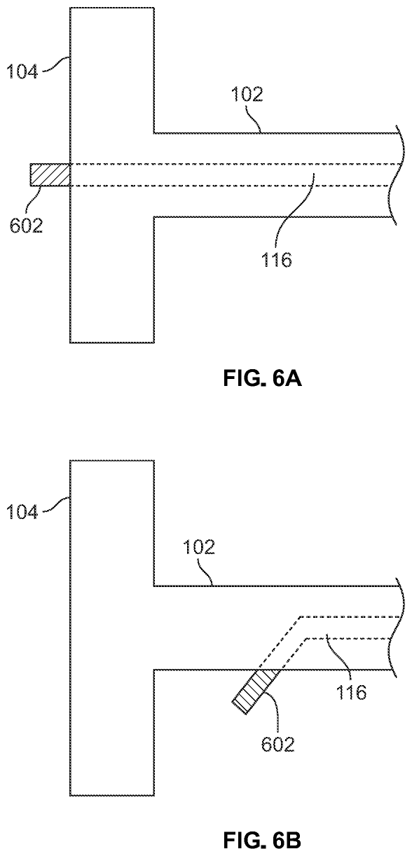

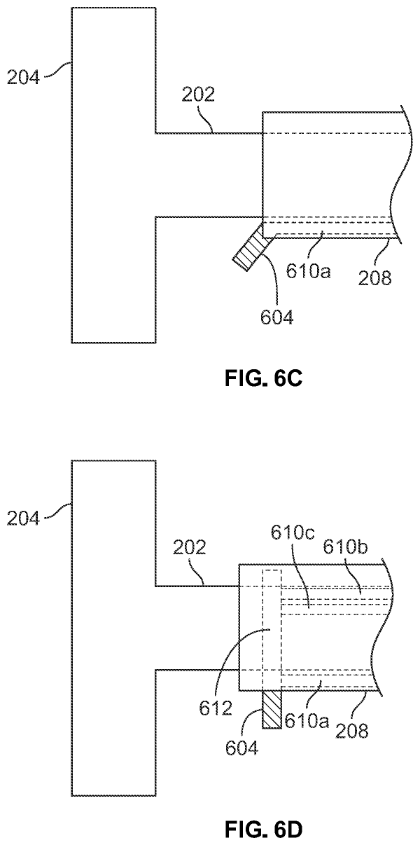

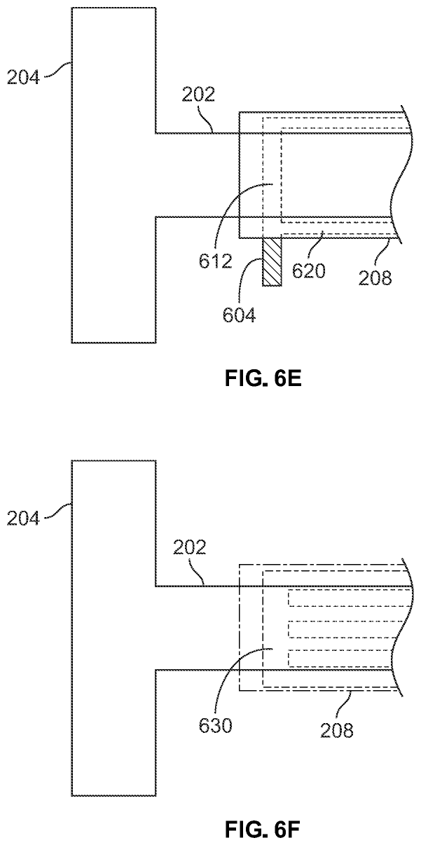

FIGS. 6A-6F are alternative embodiments of a fluid connection for the surgical tunneling tool of FIGS. 1 and 2. The example fluid connections of FIGS. 6A-6F may connect to a syringe or other external fluid source. Any of the fluid connections illustrated in FIGS. 6A-6F can be implemented in conjunction with the applicable embodiments of FIGS. 3A-3V and 5A-5D as would be understood by a person of ordinary skill in the art.

FIGS. 6A and 6B are embodiments of fluid connections for the surgical tunneling tool 100 as shown in FIG. 1. As shown in FIG. 6A, the fluid connector 602 (e.g., coupling mechanism) is disposed on the handle 104 and in fluid communication with the lumen 116 centrally disposed within the flexible shaft 102. In contrast, as shown in FIG. 6B, the fluid connector 602 is disposed on the flexible shaft 102 but still in fluid communication with the lumen 116. As shown in FIG. 6A, the fluid connector 602 is disposed perpendicular to the handle 104 and in FIG. 6B, the fluid connector 602 is disposed oblique to the flexible shaft 102. Alternatively, the fluid communication may be disposed at any angle relative the handle and/or flexible shaft.

In the example of FIGS. 6A-6F, the fluid connector 602 can include any known fluid connection. The fluid connector 602 may include a luer lock, threaded connection, press-fit connection, flange connection, etc. The fluid connector 602 may also be configured for fluid connection with specific external fluid sources, e.g., syringes. Additionally, the fluid connection can include an external pressure source (e.g., syringe, peristaltic pump, pneumatic pump, etc.) capable of pressurizing fluid disposed in the lumen 116. Pressurizing fluid disposed in the lumen 116 from the fluid connector 602 causes fluid to be expelled through an orifice, in accordance with the present disclosure.

FIGS. 6D-6E illustrate example coupling mechanism 604 (e.g., fluid connector) for the surgical tunneling tool 200 as shown in FIG. 2. As shown in FIGS. 6c and 6d, the coupling mechanism 604 is in fluid communication with at least one fluid channel disposed within the sheath 208. For example, as shown in FIG. 6C, the coupling mechanism 604 is disposed in fluid connection with one channel 610a. In contrast the coupling mechanism 604 of FIG. 6D is in fluid connection with three fluid channels 610a, 610b, and 610c via an annular fluid channel 612. In the example of FIG. 6D, the annular fluid channel 612 distributes fluid from the coupling mechanism 604 between the three fluid channels 610a, 610b, and 610c. Further, as shown in FIG. 6E, the coupling mechanism 604 connects with an open space disposed along a length of the sheath 208. In such embodiments, the sheath 208 the fluid storage volume of the sheath 208 is increased.

FIG. 6F provides an embodiment of the surgical tunneling tool 200 as shown in FIG. 2 without a fluid connection at the proximal end of the sheath. In various embodiments, the channels 630 of the sheath 208 can be filled prior to inserting the surgical tunneling tool 200 into a patient via at least one orifice disposed on the distal end of the surgical tunneling tool. In other embodiments, the sheath 208 may be compressible and pressurize the fluid stored within the sheath 208 as the surgical tunneling tool 200 is inserted into the patient. Accordingly, fluid disposed in the channels 630 can be released from the sheath 208 proportionally to the extent the surgical tunneling tool 200 is inserted into the patient.

As shown in the embodiments of FIGS. 3A-3V, 5A-5D, and 6A-6F are shown with lumens disposed in the flexible shaft 102, 202, 402 or channels disposed in the sheath 208, 540. Yet, in accordance with the present disclosure, each of the surgical tunneling tools 100, 200, and 400 can include lumens disposed in the flexible shaft 102, 202, 402 and also include channels disposed in the sheath 208, 540. In such embodiments, the surgical tunneling tool 100, 200, 400 may also include multiple internal containers 434, external fluid sources and fluid connections 142, 242, or 432. As a result, it should be understood that two or more of the embodiments of FIGS. 3A-3V, 5A-5D, and 6A-6F can be combined to form additional embodiments of the surgical tunneling tool 100, 200, 400. In such examples, the lumen disposed in the flexible shaft 102, 202, 402 may have a first fluid (e.g., anesthetic having a first concentration, lubricant, saline, etc.) and the sheath 208, 540 may have a second fluid (e.g., anesthetic having a second concentration, lubricant, saline, etc.). Further, each of the flexible shaft 102, 202, 402 and the sheath 208, 540 can include a plurality of orifices in fluid communication with corresponding lumens and channels. In such examples, a subset of orifices on one or each of the flexible shaft 102, 202, 402 and the sheath 208, 540 include a different fluid or are configured to remove fluid from a subdermal pathway.

The construction of the surgical tunneling tools and corresponding methods of use are illustrative only. While only a few embodiments have been described in detail, the surgical tunneling tool of the present disclosure may involve various modifications. In some embodiments, variations in size, relative dimensions, shapes, mounting arrangements, use of materials, orientations, etc. are considered within the teachings of the present disclosure. Further, in other embodiments, the position of elements may be reversed or otherwise varied in position or quantity. Further, the order or sequence of any process or steps may be altered or re-sequenced according to alternative embodiments but remain within the teachings of the present disclosure. Other substitutions, modifications, changes, and omissions may be made to the construction of the surgical tunneling tool or the method of use without departing from the scope of the present disclosure.

1-78. (canceled)

79. A surgical tunneling tool, comprising:
a flexible shaft, defining a first lengthwise axis, including a lumen disposed along a length of the first lengthwise axis of the flexible shaft, the flexible shaft having a proximal end and a distal end;
a handle disposed at the proximal end of the flexible shaft;
a tip disposed at the distal end of the flexible shaft; and
at least one orifice, disposed proximate the distal end of the flexible shaft, in fluid communication with the lumen.

80. The surgical tunneling tool of claim 79, wherein the flexible shaft is flat.

81. The surgical tunneling tool of claim 79, wherein the at least one orifice in fluid communication with the lumen is disposed on the tip.

82. The surgical tunneling tool of claim 79, wherein the flexible shaft includes a connector, in fluid communication with the lumen, configured to receive fluid from an external fluid source.

83. The surgical tunneling tool of claim 79, wherein the handle includes an actuator to selectively pressurize fluid in the lumen.

84. The surgical tunneling tool of claim 83, wherein the actuator comprises at least one of: a lever; a button; a switch; a plunger; and a trigger.

85. The surgical tunneling tool of claim 84, wherein the actuator actuates at least one of: a mechanical pump; an electrical pump; peristaltic pump; and pneumatic device.

86. The surgical tunneling tool of claim 79, wherein the tip is one of a Hasson trocar and a blunt trocar.

87. The surgical tunneling tool of claim 79, further including a sheath, movable relative to the flexible shaft, defining a second lengthwise axis, disposed along a second length of the flexible shaft, wherein the first lengthwise axis and the second lengthwise axis are parallel.

88. The surgical tunneling tool of claim 87, wherein the sheath disposed along a length of the flexible shaft includes a distal end disposed on the flexible shaft proximate the tip.

89. The surgical tunneling tool of claim 88, further including echogenic particles disposed in at least one of the flexible shaft and the sheath.

90. The surgical tunneling tool of claim 79, wherein the tip is porous.

91. The surgical tunneling tool of claim 79, wherein each orifice of the at least one orifice defines a central axis disposed centrally within the orifice.

92. The surgical tunneling tool of claim 91, wherein the central axis of at least one orifice is either parallel or perpendicular with the first lengthwise axis of the flexible shaft.

93. The surgical tunneling tool of claim 87, wherein the flexible shaft includes a plurality of orifices.

94. The surgical tunneling tool of claim 93, wherein the plurality of orifices are disposed on the distal end of the flexible shaft proximate the tip.

95. The surgical tunneling tool of claim 79, including at least one container disposed in the handle, the at least one container in fluid communication with the lumen.

96. The surgical tunneling tool of claim 95, wherein the handle includes a first container in fluid communication with a first lumen and a first orifice and a second container in fluid communication with a second lumen and a second orifice.

97. A method comprising:
using the surgical tunneling tool of claim 79 to create, in biological tissue, a space for insertion of a medical device.

98. The method of claim 97, wherein the method comprises stepwise insertion of the surgical tunneling tool.
